(19) United States
(12) Patent Application Publication
(10) Pub. No.: US 2021/0361851 A1
(43) Pub. Date: Nov. 25, 2021

(54) DEVICE FOR CLEANING LIQUID FROM AN EYE OF A SUBJECT

(71) Applicant: I-MED PHARMA INC., Dollard des Ormeaux (CA)

(72) Inventor: Zvi Nachum, Tiberias (IL)

(21) Appl. No.: 17/291,171

(22) PCT Filed: Nov. 1, 2019
(86) PCT No.: PCT/IB2019/059387

§ 371 Date: May 4, 2021

(30) Foreign Application Priority Data
Nov. 8, 2018 (GB) 1818192.5

(51) Int. Cl. A61M 1/00

(52) U.S. Cl. CPC A61M 1/774; A61M 1/76; A61M 2210/0612; A61M 2205/8206; A61M 2209/084; A61M 2205/7536

(57) ABSTRACT

A device for removing a liquid, such as mucus, from an eye of a subject using sub-atmospheric pressure. The device includes a handle and a removable head, separated by a filter, such that when mucus is removed from the eye into the removable head, the handle remains clean.

This application draws priority from GB Patent Application No. GB1818192.5, filed Nov. 8, 2018, which application is incorporated by reference for all purposes as if fully set forth herein.

### FIELD OF THE INVENTION

The present invention relates to an instrument for removing liquid from an eye of a subject, and more specifically, to an instrument having a removable head for removing liquid, such as mucus, from an eye of a subject using vacuum while maintaining a handle of the instrument sterile.

### SUMMARY OF THE INVENTION

According to an aspect of a first embodiment of the teachings of the present invention there is provided a device for cleaning liquid from an eye of a subject, the device including:

a removable head, including a first hollow passage disposed along a length of the removable head, a tip dimensioned to contact the eye of the subject, and a base portion;

a body portion including an upper rim removably engaging the base portion of the removable head and a lower portion, the body portion including a second hollow passage;

a filter disposed between the tip of the removable head and the body portion,

wherein a fluid flow path is formed between the first hollow passage and the second hollow passage via the filter,

wherein, in an operative mode of the device, when sub-atmospheric pressure is formed in the body portion and the tip of the removable head contacts the eye of the subject, liquid is drawn from the eye of the subject toward the body portion via the fluid flow path, the filter preventing the liquid from entering the body portion.

In some embodiments, the lower portion of the body portion is dimensioned and configured to be hand-held by an operator. In some embodiments, the lower portion of the body portion is elongate.

In some embodiments, the removable head is removable from the body portion without damaging functionality of the body portion.

In some embodiments, the device further includes a reuse identifying or preventing mechanism adapted to identify removal of the removable head from the body portion, and to identify or prevent reattachment of the removable head to the body portion, thereby to identify or prevent reuse of the removable head. In some embodiments, the reuse identifying or preventing mechanism is adapted to provide an alert to the operator upon identification of reattachment of the removable head to the body portion, the alert perceivable by the operator.

In some embodiments, the removable head is disposable following a single use thereof.

In some embodiments, the removable head is reversibly removable from the body portion, using at least one of click-fit engagement, snap fit engagement, magnetic engagement, or threaded engagement.

In some embodiments, the removable head includes a seat adjacent the base portion, the seat adapted to receive the filter, wherein the filter is removable from the body portion together with the removable head.

In some embodiments, the filter is welded into the seat.

In some embodiments, the filter is pressured into the seat.

In some embodiments, the filter is adapted to absorb a liquid volume of up to 0.5 ml.

In some embodiments, the filter may have a thickness in the range of 2-20 mm.

In some embodiments, the filter may have a diameter in the range of 5-40 mm.

In some embodiments, the device further includes a cap connected to the base portion of the removable head, the cap including a hollow in fluid communication with the first hollow passage of the removable head and forming part of the fluid flow path, the cap adapted to engage the upper rim of the body portion, wherein the filter is disposed within the removable head between the tip and the cap.

In some embodiments, the body portion is functionally associated with a sub-atmospheric pressure generating mechanism in fluid communication with the first hollow of the removable head, the sub-atmospheric pressure generating mechanism adapted, in the operative mode, to form the sub-atmospheric pressure in the body portion thereby to draw the liquid from the eye of the user.

In some embodiments, the sub-atmospheric pressure generating mechanism includes at least the lower portion of the body portion being flexible and compressible by the operator to form sub-atmospheric pressure therein.

In some embodiments, the sub-atmospheric pressure generating mechanism includes a mechanical vacuum generator. In some embodiments, the mechanical vacuum generator is disposed at least partially within the body portion.

In some embodiments, the sub-atmospheric pressure generating mechanism includes an electric vacuum generator.

In some embodiments, the electric vacuum generator is disposed within the body portion. In some embodiments, the device further includes a battery disposed within the body portion and adapted to power the electric vacuum generator.

In some embodiments, the electric vacuum generator is external to the body portion and is connected thereto by a tube.

In some embodiments, the tip of the removable head includes a plurality of appendages shaped and dimensioned to contact the eye of the subject.

In some embodiments, the plurality of appendages each terminates in a rounded surface.

In some embodiments, the plurality of appendages are flexible.

In some embodiments, the plurality of appendages are resilient.

In some embodiments, at least the plurality of appendages are formed of a medical grade silicone.

In some embodiments, at least the plurality of appendages have a Shore A hardness in the range of 20 to 50.

In some embodiments, an obtuse angle is formed between the tip of the removable head and the lower portion of the body portion.

In some embodiments, the body portion is straight along a longitudinal axis thereof, and the removable head is angled to form the obtuse angle.

In some embodiments, the removable head is straight along a longitudinal axis thereof, and the body portion is angled to form the obtuse angle.

In some embodiments, the subject is a human subject. In some embodiments, the operator is the subject.

In some embodiments, the operator is a person other than the subject.

In some embodiments, the liquid includes mucus.

According to another aspect of the first embodiment of the teachings of the present invention there is provided a method of removing liquid from an eye of a subject using the device of the first embodiment described herein, the method including:

contacting the tip of the removable head with a surface of the eye of the subject; and

operating the device to form sub-atmospheric pressure in the body portion, thereby to draw the liquid from the eye of the subject toward the body portion.

According to an aspect of a second embodiment of the teachings of the present invention there is provided a method of removing liquid from an eye of a subject, the method including:

providing a liquid cleaning device including:
a removable head, including a first hollow passage disposed along a length of the removable head, a tip dimensioned to contact the eye of the subject, and a base portion;
a body portion including an upper rim removably engaging the base portion of the removable head and a lower portion, the body portion including a second hollow passage; and
a filter disposed between the tip of the removable head and the body portion,
wherein a fluid flow path is formed between the first hollow passage and the second hollow passage via the filter,

contacting the tip of the removable head with a surface of the eye of the subject; and

operating the device to form sub-atmospheric pressure in the body portion, thereby to draw the liquid from the eye of the subject toward the body portion.

In some embodiments, the method further includes, following the operating the device, removing the removable head from the body portion.

In some embodiments, the method further includes following the removing the removable head, disposing of the removable head.

In some embodiments, the method further includes, following the removing the removable head, sterilizing the removable head.

In some embodiments, the method further includes, prior to the contacting, connecting a sterile the removable head to the body portion.

In some embodiments, the liquid includes mucus.

In some embodiments, the sub-atmospheric pressure in the body portion is sufficient to generate a sub-atmospheric pressure, at the surface of the eye, of at least 0.001 psi, at least 0.01 psi, at least 0.1 psi, at least 0.3 psi, or at least 0.5 psi, and optionally, at most 5 psi, so as to draw said liquid into the first hollow of the removable head.

In some embodiments, the sub-atmospheric pressure in the body portion is sufficient to generate a sub-atmospheric pressure, at the surface of the eye, of at most 3 psi, at most 2 psi, or at most 1 psi, so as to draw said liquid into the first hollow of the removable head.

According to an aspect of a third embodiment of the teachings of the present invention there is provided a device for cleaning liquid from an eye of a subject, the device including:

a head, including a first hollow passage disposed along a length of the head, and a tip dimensioned to contact the eye of the subject, and a base portion; and

a body portion including an upper rim engaging the base portion of the head and a lower portion, the body portion including a second hollow passage in fluid communication with the first hollow passage, wherein a fluid flow path is formed between the first hollow passage and the second hollow passage, wherein, in an operative mode of the device, when a vacuum generator is functionally associated with the body portion and in fluid communication with the second hollow passage and the tip of the head contacts the eye of the subject, the device is adapted to draw liquid from the eye of the subject toward the body portion via the fluid flow path.

In some embodiments, the lower portion of the body portion is dimensioned and configured to be hand-held by an operator.

In some embodiments, the lower portion of the body portion is elongate.

In some embodiments, the head includes a removable head, such that the base portion of the removable head is adapted to removably engage the upper rim of the body portion.

In some embodiments, the removable head is removable from the body portion without damaging functionality of the body portion.

In some embodiments, the device further includes a reuse identifying or preventing mechanism adapted to identify removal of the removable head from the body portion, and to identify or prevent reattachment of the removable head to the body portion, thereby to identify or prevent reuse of the removable head.

In some embodiments, the reuse identifying or preventing mechanism is adapted to provide an alert to the operator upon identification of reattachment of the removable head to the body portion, the alert perceivable by the operator.

In some embodiments, the removable head is disposable following a single use thereof.

In some embodiments, the removable head is reversibly removable from the body portion, using at least one of click-fit engagement, snap fit engagement, magnetic engagement, or threaded engagement.

In some embodiments, the device further includes a filter disposed between the tip of the removable head and the body portion, such that the fluid flow path is formed between the first hollow passage and the second hollow passage via the filter, and wherein, when liquid is drawn from the eye of the subject toward the body portion, the filter prevents the liquid from entering the body portion.

In some embodiments, the removable head includes a seat adjacent the base portion, the seat adapted to receive the filter, wherein the filter is removable from the body portion together with the removable head.

In some embodiments, the filter is welded into the seat. In some embodiments, the filter is pressured into the seat.

In some embodiments, the filter is adapted to absorb a liquid volume of up to 0.5 ml.

In some embodiments, the device further includes a cap connected to the base portion of the removable head, the cap including a hollow in fluid communication with the first hollow passage of the removable head and forming part of the fluid flow path, the cap adapted to engage the upper rim of the body portion, wherein the filter is disposed within the removable head between the tip and the cap.

In some embodiments, the vacuum generator includes a mechanical vacuum generator.

In some embodiments, the vacuum generator includes an electric vacuum generator.

In some embodiments, the electric vacuum generator is disposed within the body portion, and wherein the device further includes a battery disposed within the body portion and adapted to power the electric vacuum generator.

In some embodiments, the vacuum generator is external to the body portion and is functionally associated therewith by a tube.

In some embodiments, the tip of the head includes a plurality of appendages shaped and dimensioned to contact the eye of the subject.

In some embodiments, the plurality of appendages each terminates in a rounded surface.

In some embodiments, the plurality of appendages are flexible.

In some embodiments, the plurality of appendages are resilient.

In some embodiments, at least the plurality of appendages are formed of a medical grade silicone.

In some embodiments, at least the plurality of appendages have a Shore A hardness in the range of 20 to 50.

In some embodiments, an obtuse angle is formed between the tip of the head and the lower portion of the body portion.

In some embodiments, the body portion is straight along a longitudinal axis thereof, and the head is angled to form the obtuse angle.

In some embodiments, the head is straight along a longitudinal axis thereof, and the body portion is angled to form the obtuse angle.

In some embodiments, the operator is the subject. In some embodiments, the operator is a different person than the subject.

In some embodiments, the liquid includes mucus.

According to another aspect of a third embodiment of the teachings of the present invention there is provided a method of removing liquid from an eye of a subject using the device of the third embodiment, the method including:

contacting the tip of the head with a surface of the eye of the subject; and

operating the vacuum generator to generate vacuum, thereby to draw the liquid from the eye of the subject toward the body portion.

According to an aspect of a fourth embodiment of the teachings of the present invention there is provided a method of removing liquid from an eye of a subject, the method including:

contacting a tip of a head of a liquid removing device with a surface of the eye of the subject, the head of the liquid removing device having a hollow passage therethrough forming part of a fluid flow path from the tip of the liquid removing device to a body portion of the liquid removing device;

generating a sub-atmospheric pressure within the body portion of the liquid removing device, thereby to draw the liquid from the eye of the subject, via the fluid flow path, toward the body portion of the liquid removing device.

In some embodiments, the head of the liquid removing device is removable, and the method further includes, prior to the contacting, connecting a sterile head to the body portion of the liquid removing device.

In some embodiments, the method further includes, following the generating the sub-atmospheric pressure, removing the removable head from the body portion, without causing damage to functionality of the body portion.

In some embodiments, the method further includes, following the removing, disposing of the removable head.

In some embodiments, the method further includes, following the removing, sterilizing the removed removable head.

In some embodiments, the head includes a filter separating the first hollow passage of the head from the body portion of the liquid removing device, and wherein during the drawing of the liquid toward the body portion, the filter prevents the drawn liquid from entering the body portion.

In some embodiments, the liquid includes mucus.

According to an aspect of a fifth embodiment of the teachings of the present invention there is provided an elongate removable head adapted for connection to a body portion of a device for cleaning liquid from an eye of a subject, the elongate removable head including.

a hollow passage disposed along a length of the removable head;

a tip dimensioned to contact the eye of the subject; and

a base portion adapted to removably engage the body portion of the device such that, when the base portion engages the body portion of the device, the hollow passage is in fluid communication with piping in the body portion of the device and forms a fluid flow path therewith,

wherein, when sub-atmospheric pressure is formed in the body portion and the tip of the removable head contacts the eye of the subject, liquid is drawn from the eye of the subject into the tip.

In some embodiments, the elongate removable head further includes a filter disposed between the tip and the base portion, wherein when liquid is drawn from the eye of the subject into the tip, the filter is adapted to prevent the liquid from entering the body portion.

In some embodiments, the elongate removable head further includes a reuse preventing mechanism, adapted, upon removal of the removable head from the base portion of the device, to provide a signal indicating that the removable head has been used, such that the removable head cannot be reused when re-connected to the body portion.

In some embodiments, the removable head is adapted to be reversibly removable from the body portion.

In some embodiments, wherein the tip of the removable head includes a plurality of appendages shaped and dimensioned to contact the eye of the subject.

In some embodiments, the plurality of appendages each terminates in a rounded surface.

In some embodiments, the plurality of appendages are flexible.

In some embodiments, the plurality of appendages are resilient.

In some embodiments, at least the plurality of appendages are formed of a medical grade silicone.

In some embodiments, at least the plurality of appendages have a Shore A hardness in the range of 20 to 50.

In some embodiments, an obtuse angle is formed between the tip and the base portion of the removable head.

In some embodiments, the liquid includes mucus.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are used to designate like elements.

In the drawings:

FIG. 1 is an exploded view illustration of a device for removing liquid, such as mucus, from an eye of a subject according to an embodiment of the present invention;

FIG. 2 is a perspective view illustration of the device of FIG. 1, when constructed and disposed in a charger;

FIG. 3 is a sectional illustration of the device of FIG. 2; and

FIGS. 4A and 4B are a perspective view illustration and a side view planar illustration of a device for removing mucus from an eye of a user, according to another embodiment of the present invention.

### DETAILED DESCRIPTION OF THE ILLUSTRATED EMBODIMENTS

The principles and operation of the inventive device for removing mucus from an eye of a subject may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

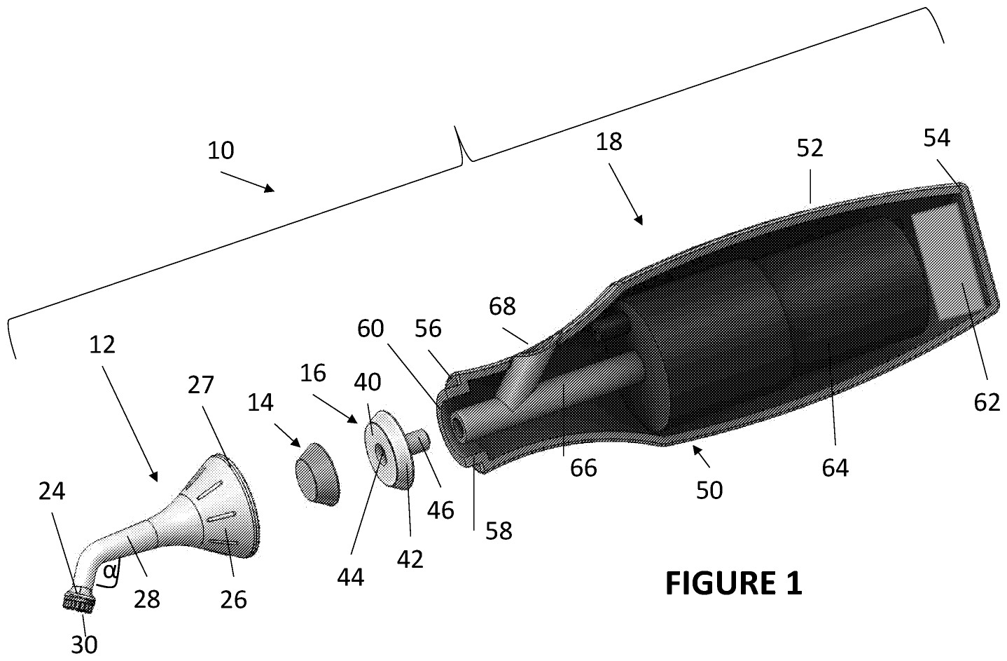

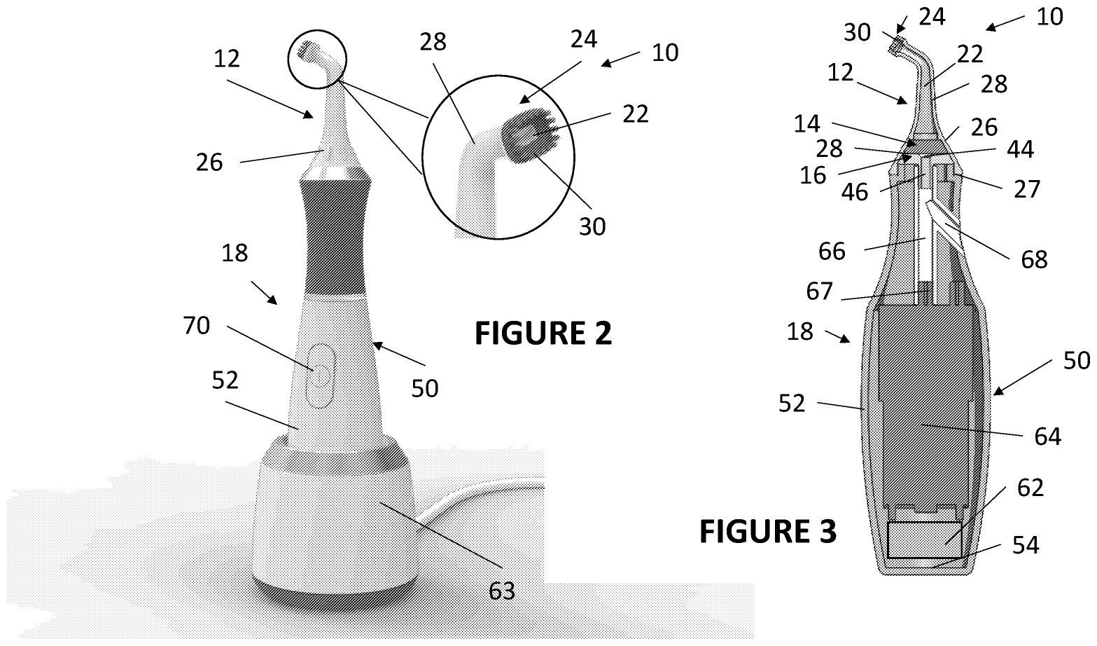

Reference is now made to FIG. 1, which is an exploded view illustration of a device 10 for removing liquid, such as mucus, from an eye of a subject according to an embodiment of the present invention, to FIG. 2, which is a perspective view illustration of the device 10, when constructed and disposed in a charger, and to FIG. 3, which is a sectional illustration of the device 10.

The device 10 includes a removable head 12, a filter 14, a cap 16 for the removable head, and a body portion 18, which is also termed “handle” herein, and may function as a handle for the device. In some embodiments, a pump, or other sub-atmospheric pressure generator 20, also termed “vacuum generator” herein, is disposed within handle 18.

The removable head 12 includes a hollow 22 disposed along a length of the removable head (FIG. 3). Removable head 12 terminates, at a proximal end thereof, in a tip 24 adapted for engaging the eye of the subject, and at a distal end in a base portion 26 adapted for connecting to handle 18. Base portion 26 terminates in a ring shaped wall portion 27. An elongate hollow neck portion 28 connects tip 24 and base portion 26.

In some embodiments, neck portion 28 is bent, to define an obtuse angle between tip 24 and base portion 26, indicated by a.

In some embodiments, tip 24 includes a plurality of flexible and resilient appendages 30, each terminating in a rounded surface adapted to engage the eye of the subject. In some such embodiments, at least the appendages 30 are formed of a medical grade silicone.

In some embodiments, removable head 12 has a Shore A hardness in the range of 20 to 50.

Filter 14 has pores suitably sized to allow air flow therethrough, and to prevent passage of mucus and liquid therethrough. In some embodiments, the filter is also adapted to absorb a liquid, for example in a quantity of up to 0.5 ml. The filter 14 is preferably dimensioned to absorb moisture from an eye of a human, and in some embodiments has a thickness in the range of 2-20 mm. In some embodiments, filter 14 has a diameter in the range of 5-40 mm.

In some embodiments, filter 14 is disposed within a dedicated seat 32 in base portion 26, as illustrated in FIG. 3. Filter 14 may be welded to the seat 32, or may be pressurized therein, so as to engage an interior surface of the seat and to prevent liquid, such as mucus, passing through hollow 22 from circumventing the filter 14. For example, in the illustrated embodiment, filter 14 comprises a truncated cone.

Cap 16 is adapted to engage base portion 26, so as to enclose filter 14 within removable head 12. Cap 16 includes a disc portion 40 having an outer surface 42, and a hollow center 44. The outer surface 42 is shaped and adapted to engage an inner surface of base portion 26 of head 12. As such, in the illustrated embodiment, outer surface 42 is tapered.

A tube 46 is in fluid flow communication with hollow center 44 and extends distally of disc portion 40. Hollow center 44 and tube 46 are in fluid flow communication with hollow 22 of head 12, via filter 14.

Handle 18 includes a shell 50, including a circumferential wall portion 52 terminating at one, distal end thereof in a base wall 54 and at the other, proximal end thereof in a shoulder 56. Extending longitudinally proximally from shoulder 56 is a cowl wall portion having a circumferential external surface 58, and terminating, at a proximal end, in a wall surface 60. In some embodiments, handle 18 has a diameter in the range of 2 cm to 5 cm.

In some embodiments, a power source 62, such as a rechargeable battery, is disposed within shell 50. In some such embodiments, circumferential wall portion 52 or base portion 54 includes an electrical contact (not shown) for electrical connection of the power source 62 to a power supply, such as a charger 63. In other embodiments, device 10 may include a suitable wire (not shown) for direct connection to a power supply, such as an electrical socket.

In some embodiments, a vacuum pump 64 is disposed within shell 50, and powered by power source 62. Extending proximally from vacuum pump 64 toward the proximal end of handle 18 is a vacuum tube 66, connected to a suitable port 67 of vacuum pump 64. In some embodiments, vacuum tube 66 includes a branched tube 68 extending out of handle 18, for connection of vacuum tube 66 to an external vacuum generator (not shown). An activation button 70 may be disposed on an external surface of circumferential wall portion 52, as seen clearly in FIG. 2. Activation button 70 is functionally associated with power source 62 and/or with vacuum pump 64.

As seen clearly in FIG. 3, a lower surface of disc 40 of cap 16 engages proximal wall portion 60 of handle 18. Simultaneously, an inner surface of base portion 26 engages circumferential portion 58 of handle 18 and wall 27 of base portion 26 engages shoulder 56 of handle 18. Tube 46 is disposed within vacuum tube 66, such that hollow 22 is in fluid flow communication with vacuum tube 66, via filter 14 and tube 46, for generation of a vacuum within removable head 12 during operation of vacuum pump 64.

However, other methods of engaging removable head 12 to handle 18 may be used, such as snap fit engagement, click fit engagement, threaded engagement, or magnetic engagement.

In some embodiments, a different sub-atmospheric pressure generator may be used to generate sub-atmospheric pressure, or a vacuum, within removable head 12. For example, the sub-atmospheric pressure may be generated by shell 50 being compressed to remove air therefrom and then released. As another example, a mechanical sub-atmospheric pressure generator may be used.

In some embodiments, the dimensions and shape of shell 50 are selected so as to be conveniently held by a human hand. In some embodiments, the location of activation button 70 is selected such that device 10 may be conveniently operated by a human operator.

In some embodiments, the device has a weight in the range of 100 g to 500 g.

In use, an operator, which may be the subject or a caregiver of the subject, engages tip 24 of removable head 12 with a surface of the eye of the subject, and activates vacuum pump 64 by depressing or otherwise operating activation button 70. Under the force of vacuum caused by pump 64, vacuum tube 66, tube 46, and filter 14, mucus is then drawn, from the surface of the eye of the user to device 10, via hollow 22 of the head 12 to filter 14.

In some cases, in which it is desirable for removable head 12 to be sterile, the operator may connect a sterile removable head 12 to handle 18 prior to engaging the tip 24 to the eye of the subject, by connecting base portion 26 to the handle as described above.

In some embodiments, following removal of mucus from the subject's eye, the removable head 12 is disconnected from handle 18. The removable head 12 may then be discarded, or may be subjected to a sterilization process, for future use of the removable head.

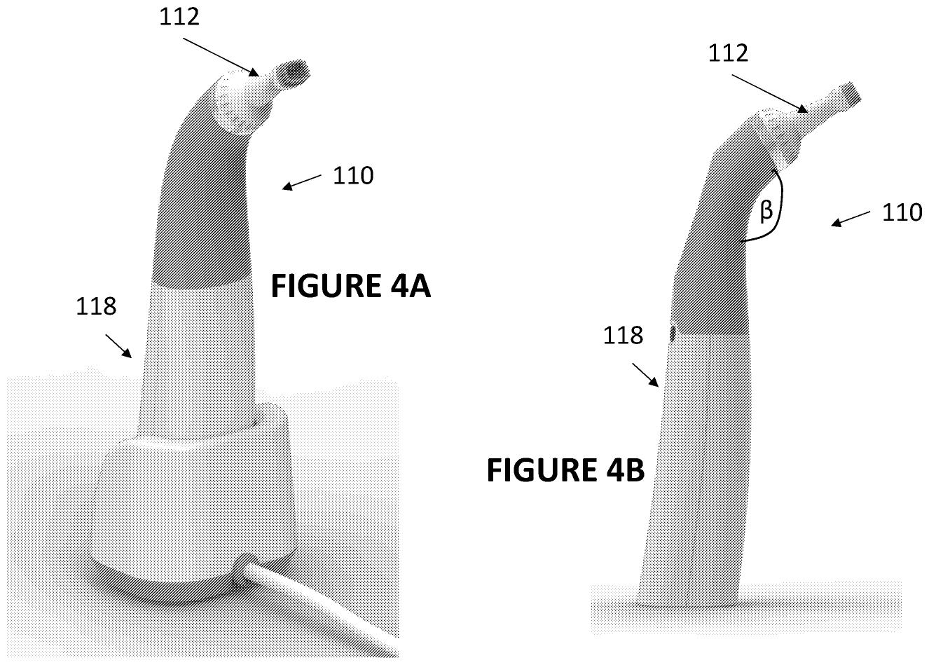

In some embodiments, removable head 12 and handle 18 each include a suitable configured electronic circuit (not explicitly shown). In some such embodiments, upon connection of removable head 12 to handle 18, the electronic circuits communicate, or create a “handshake”. During such a handshake, the electronic circuit of removable head 12 receives a signal, or is otherwise notified, that the removable head has been connected to the handle. Should a user try to reuse the same removable head 12, upon re-connection of removable head 12 to a handle the handshake between the removable head and the handle will fail, since the electronic circuit of the removable head will indicate that the removable head has already been used. The user may be alerted to this situation by a perceivable alert, such as a sound or a visual alert, or alternatively the sub-atmospheric pressure generator may be disabled until there is a valid handshake between the removable head and the handle. Reference is now made to FIGS. 4A and 4B, which, respectively, are a perspective view illustration and a side view planar illustration of a device 110 for removing mucus from an eye of a user, according to another embodiment of the present invention. Reference numerals in FIGS. 4A and 4B, indicate the same portion of device 110 as corresponding reference numerals in FIGS. 1 to 3.

The device 110 of FIGS. 4A and 4B is substantially identical to the device 10 of FIGS. 1 to 3, and differs from device 10 only in the geometrical arrangement of the handle 118 and removable head 112. More specifically, in both devices 10 and 110 there is a bend in the device, which makes it easier for the user to reach an appropriate angle for engaging the eye. However, whereas in the device 10 of FIGS. 1 to 3 the removable head is angled, in the device 110 of FIGS. 4A and 4B, removable head 112 is relatively short, and straight, and handle 118 is bent at a top third thereof, forming an obtuse angle, indicated by β.

In all other aspects, device 110 is substantially identical to device 10, and has similar components and a similar function.

As used herein in the specification and in the claims section that follows, the term “subject” relates to a mammalian subject, including a human.

As used herein in the specification and in the claims section that follows, the term “operator” relates to a human operator, who holds the device of the present invention during operation thereof.

As used herein in the specification and in the claims section that follows, the term “caregiver” relates to a human who takes care of a subject, such as a doctor, a veterinarian, a medical practitioner, a parent, and a pet-owner.

As used herein in the specification and in the claims section that follows, the term “disposable” relates to an object or element which is designed to be used in sterile conditions, and to be discarded of following a single use thereof, and/or that cannot be sterilized and thus must be discarded after a single use thereof.

As used herein in the specification and in the claims section that follows, the term “removable” relates to a first object or element which is designed to be removed from a second object or element to which it is connected without damaging the second object or element, regardless of whether or not the first object or element can be reattached to the second object or element.

As used herein in the specification and in the claims section that follows, the term “reversibly removable” relates to a first object or element which is designed to be removable from a second object or element, and can be reattached to the second object or element. Connections using threaded engagement, magnetic engagement, and snap fit engagement may all allow for a reversibly removable connection between two elements.

As used herein in the specification and in the claims section that follows, the term “handheld” relates to an object which can conveniently be held in a single hand of a human adult. In some cases, a handheld object may be connected to another, non-handheld object, for example by means of a tube or a wire. For example, a handheld object has a weight of at most 2 kg, at most 1.5 kg, at most 1 kg, at most 800 g, at most 700 g, at most 600 g, at most 500 g, at most 300 g, at most 200 g, or at most 100 g. As another example, a handheld object has a diameter in the range of 1 mm to 20 cm, in the range of 1 mm to 15 cm, or in the range of 5 mm to 10 cm.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A device for cleaning liquid from an eye of a subject, the device comprising:
a removable head, including a first hollow passage disposed along a length of said removable head, a tip dimensioned to contact the eye of the subject, and a base portion;
a body portion including an upper rim removably engaging said base portion of said removable head and a lower portion, said body portion including a second hollow passage;
a filter disposed between said tip of said removable head and said body portion,
wherein a fluid flow path is formed between said first hollow passage and said second hollow passage via said filter,
wherein, in an operative mode of said device, when sub-atmospheric pressure is formed in said body portion and said tip of said removable head contacts the eye of the subject, liquid is drawn from the eye of the subject toward said body portion via said fluid flow path, said filter preventing said liquid from entering said body portion.

2. The device of claim 1, wherein said lower portion of said body portion is dimensioned and configured to be hand-held by an operator, and said lower portion of said body portion is elongate.

3. The device of claim 1, wherein said removable head is removable from said body portion without damaging functionality of said body portion.

4. The device of claim 1, said device further including a reuse identifying or preventing mechanism adapted to identify removal of said removable head from said body portion, and to identify or prevent reattachment of said removable head to said body portion, thereby to identify or prevent reuse of said removable head.

5. The device of claim 4, wherein said reuse identifying or preventing mechanism is adapted to provide an alert to the operator upon identification of reattachment of said removable head to said body portion, said alert perceivable by said operator.

6. The device of claim 1, wherein said removable head is reversibly removable from said body portion, using at least one of click-fit engagement, snap fit engagement, magnetic engagement, or threaded engagement.

7. The device of claim 1, wherein said removable head includes a seat adjacent said base portion, said seat adapted to receive said filter, wherein said filter is removable from said body portion together with said removable head.

8. The device of claim 1, wherein said filter is adapted to absorb a liquid volume of up to 0.5 ml.

9. The device of claim 1, wherein said filter has at least one of: a thickness in the range of 2-20 mm and a diameter in the range of 5-40 mm.

10. The device of claim 1, further comprising a cap connected to said base portion of said removable head, said cap including a third hollow passage in fluid communication with said first hollow passage of said removable head and forming part of said fluid flow path, said cap adapted to engage said upper rim of said body portion, wherein said filter is disposed within said removable head between said tip and said cap.

11. The device of claim 1, wherein said body portion is functionally associated with a sub-atmospheric pressure generating mechanism in fluid communication with said first hollow of said removable head, said sub-atmospheric pressure generating mechanism adapted, in said operative mode, to form said sub-atmospheric pressure in said body portion thereby to draw the liquid from the eye of the user.

12. The device of claim 11, wherein said sub-atmospheric pressure generating mechanism comprises at least said lower portion of said body portion being flexible and compressible by said operator to form said sub-atmospheric pressure therein.

13. The device of claim 11, wherein said sub-atmospheric pressure generating mechanism comprises a mechanical vacuum generator, which is disposed at least partially within said body portion.

14. The device of claim 11, wherein said sub-atmospheric pressure generating mechanism comprises an electric vacuum generator.

15. The device of claim 14, wherein said electric vacuum generator is disposed within said body portion.

16. The device of claim 15, further comprising a battery disposed within said body portion and adapted to power said electric vacuum generator.

17. The device of claim 14 wherein said electric vacuum generator is external to said body portion and is connected thereto by a tube.

18. The device of claim 1, wherein said tip of said removable head comprises a plurality of appendages shaped and dimensioned to contact the eye of the subject.

19. The device of claim 18, wherein said plurality of appendages each terminates in a rounded surface, and each of the plurality of appendages is at least one of flexible and resilient.

20. The device of claim 18 or claim 19, wherein at least said plurality of appendages have a Shore A hardness in the range of 20 to 50.

21. The device of claim 1, wherein an obtuse angle is formed between said tip of said removable head and said lower portion of said body portion.

22. The device of claim 21, wherein said body portion is straight along a longitudinal axis thereof, and said removable head is angled to form said obtuse angle.

23. The device of claim 21, wherein said removable head is straight along a longitudinal axis thereof, and said body portion is angled to form said obtuse angle.

24. The device of claim 1, wherein said liquid comprises mucus.
